(19) United States
(12) Patent Application Publication
(10) Pub. No.: US 2021/0361201 A1
(43) Pub. Date: Nov. 25, 2021

(54) COMPUTATIONAL METHOD FOR AND SYSTEM FOR PREDICTING BIOMARKERS USING SHORT-TIME ASSESSMENTS OF CARDIAC DYNAMICS

(71) Applicant: The University of North Carolina at Greensboro, Greensboro, NC (US)

(72) Inventors: Nathaniel T. Berry, Baltimore, MD (US); Laurie Wideman Gold, Greensboro, NC (US)

(21) Appl. No.: 17/327,000

(22) Filed: May 21, 2021

Related U.S. Application Data
(60) Provisional application No. 63/028,266, filed on May 21, 2020.

(51) Int. Cl. A61B 5/145; A61B 5/024; A61B 5/0245; A61B 5/352; A61B 5/0205

(52) U.S. Cl. CPC A61B 5/14546; A61B 5/02405; A61B 5/0205; A61B 5/352; A61B 5/14532; A61B 5/0245

(57) ABSTRACT

In an aspect, a method of predicting biomarker concentrations from changes in short-time cardiac dynamics is described herein comprising measuring heart rate variability of a subject at rest; measuring heart rate variability of the subject during physical exertion; identifying changes in the heart rate variability at rest verses during physical exertion, and determining an estimated level of a biomarker present in the subject based on the identified changes.

### RELATED APPLICATION DATA

The present application claims priority pursuant to 35 U.S.C. § 119(e) to U.S. Provisional Patent Application No. 63/028,266 filed May 21, 2020 which is incorporated herein by reference in its entirety.

### BACKGROUND

Alterations in the temporal organization and synchronous patterns among physio-logic systems have been detected across the spectrum of biological systems. These dynamic relations between biomarkers provide important information about physiologic function. However, these patterns are not easily observed and difficult to characterize with traditional measures. For example, assessing blood biomarkers is difficult to achieve in real-time and usually requires a blood sample to be obtained, processed, and often stored, before the sample is finally analyzed. Blood samples taken intermittently can provide information about the mean concentration of specific markers at that point in time. However, this is an invasive method that fails to provide any information about the dynamics of these biomarkers, biomarker interactions, or future biomarker concentrations. There is a need to evolve existing approaches or develop and investigate new approaches that can provide knowledge about changes in the time-dependent regulatory behaviors of physiologic systems.

### SUMMARY

In an aspect, a method of predicting biomarker concentrations from changes in short-time cardiac dynamics comprises measuring heart rate variability of a subject at rest; measuring heart rate variability of the subject during physical exertion; identifying changes in the heart rate variability at rest verses during physical exertion, and determining an estimated level of a biomarker present in the subject based on the identified changes.

In some embodiments, measuring heart rate variability comprises recording an electrocardiogram of the subject. In more specific embodiments, measuring heart rate variability comprises measuring R-R intervals of the subject.

Methods described herein can further comprise predicting future biomarker concentrations present in the subject based on fluctuations and/or patterns in the R-R interval for a given period of time in some instances.

In some cases, the biomarker is a regulatory biomarker. Exemplary regulatory biomarker can comprise one or more of growth hormone, glucagon, insulin, nesfatin-1, galanin, cortisol, glucose, insulin-like growth factor 1, or any combination thereof. In some embodiments, the biomarker comprises one or more of glucagon, glucose, insulin, and cortisol.

In some embodiments, the biomarker is a hypothalamic-pituitary function-related biomarker. Exemplary hypothalamic-pituitary function biomarker can include a pituitary hormone comprising: a growth hormone, somatostatin, growth-hormone releasing hormone, follicle-stimulating hormone, adrenocorticotropic hormone, thyroid-stimulating hormone, luteinizing hormone, vasopressin, oxytocin, and/or gonadotropin-releasing hormone.

The biomarker can be regulated by changes in hypothalamic-pituitary hormones in some instances. In some embodiments, the hypothalamic-pituitary hormones comprise a growth hormone, somatostatin, growth-hormone releasing hormone, follicle-stimulating hormone, adrenocorticotropic hormone, thyroid-stimulating hormone, luteinizing hormone, vasopressin, oxytocin, and/or gonadotropin-releasing hormone.

In methods described herein, an estimated level of the biomarker is a blood or salivary concentration of the biomarker in the subject. In some embodiments, the estimated level of the biomarker is determined non-invasively.

In some embodiments, examining changes in the heart rate variability involves examining cardiac regulatory dynamics comprising time-domain, frequency-domain, and/or complexity (nonlinear dynamics) of heart rate variability.

Methods described herein can further comprise estimating biomarker dynamics. Biomarker dynamics can comprise output and secretory dynamics of the biomarker. In some cases, biomarker dynamics comprises output/concentration, pulse amplitude, and/or pulse burst frequency based on patterns in R-R intervals.

### BRIEF DESCRIPTION OF THE FIGURES

FIG. 1A is a timeline showing the screening visit of a participant.

FIG. 1B is a timeline showing the at-rest profile visit of a participant where blood was sampled from an IV catheter at the given intervals and RR-recording was sampled continuously for 24 hours.

FIGS. 2A and 2B are tables showing correlations among Demographic, Biomarker, and HRV indices.

FIGS. 3A-3C show an exercise study design, with FIG. 3A showing a screening visit of a participant; FIG. 3B showing the profile visit of the participant where blood was sample from an IV catheter at the given intervals, saliva collection was performed at given intervals, and RR-recording was sampled continuously; and FIG. 3C shows the exercise protocol that was completed on a cycle ergometer.

FIG. 4A is a graph showing a mean±se 24-hr GH Profiles During Exercise.

FIG. 4B is a graph showing a mean±se 24-hr GH Profiles During Rest.

FIG. 5A is a graph showing a mean±se 24-hr cortisol Profiles During Exercise.

FIG. 5B is a graph showing a mean±se 24-hr cortisol Profiles During Rest.

FIGS. 6A-6F show graphs of mean±se 24-hr HRV_{EP} Profiles During Exercise and Rest.

FIGS. 7A-7E show five iterations of a single exercise profile of a good-performing LSTM network trained on the exercise profile.

FIGS. 8A-8E show five iterations of a single rest profile of a poor-performing LSTM network trained on the rest profile.

### DETAILED DESCRIPTION

Embodiments described herein can be understood more readily by reference to the following detailed description and examples. Elements, apparatus and methods described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

Dynamical systems form the foundation of nonlinear methods of signal analysis and can be defined as systems whose state is definable though time-series that can be assessed and described by a set of mathematical laws. Assessing the dynamics of biomarkers, and/or the physiologic systems the biomarkers represent, typically requires serial sampling of blood or saliva, which can be time-consuming and cost prohibitive. Once these samples have been processed, they form a time-series that can be analyzed in a variety of ways. In some cases, electrocardiograms can be processed to extract the RR-interval to provide information about autonomic nervous system (ANS) input to the heart. These data are often analyzed using time-domain and frequency-domain methods; however, nonlinear dynamics has become an increasingly popular method of quantifying the dynamics of the RR-interval in recent years. Changes in the variability and complexity of cardiac regulation can be observed following acute perturbations such as physical stresses (e.g. exercise) as well as psychological tasks and are also associated with increased disease risk and mortality. Similarly, changes in the dynamics of the secretion of key regulatory biomarkers (e.g. growth hormone from the anterior pituitary) are also observed in response to acute perturbations such as exercise and chronic adaptations related to disease and aging. In methods described in more detail herein, changes in RR-intervals are used to monitor such changes in dynamics, and to predict levels of different biomarkers based on such changes.

The hypothalamus is often considered the key center of physiologic regulation. Thus, biomarkers with proximal hypothalamic regulatory inputs can be indicative of physiologic functioning. On such biomarker is human growth hormone (GH), which is secreted from the anterior pituitary and is regulated through growth hormone releasing hormone and somatostatin from the hypothalamus. Both the hypothalamus and the anterior pituitary receive inputs from higher order brain centers, as well as from short and long feedback loops from throughout the periphery. These inputs, coupled with the relatively short half-life, contribute to pulsatile secretory patterns of growth hormone. Changes in these secretory dynamics correlate with changes in cardiac regulatory dynamics following disease and/or improvements in health/fitness. Thus, the secretory patterns of GH can not only provide information about the dynamics of the hypothalamic-pituitary axis, but also the state of the entire physiologic system. This information is unobserved by single-point measures and low sampling frequencies, such as those commonly used in blood sampling methods. Similarly, there is an abundance of information imbedded within the changes in the normal RR-intervals that is not observed through heart rate (HR) alone. As described herein, measures of HR variability (HRV) assess changes in the cardiac control that are representative of acute and chronic, physical, social, and psychophysiologic stresses.

The general method described herein utilizes changes in cardiac regulation (assessed through the R-R interval) to predict future biomarker concentrations. A further object of this method is to provide an estimation of biomarker output (e.g. total secretion as calculated through area under the curve). A still further object of this method is to provide an estimation of biomarker dynamics (e.g. secretory dynamics: including but not limited to pulse amplitude and pulse burst frequency).

The method used to predict changes in biomarker concentrations uses a neural network to learn how changes in short-time indices of cardiac regulation are associated with changes in biomarker concentrations to predict future biomarker concentrations based on observed changes in cardiac dynamics. This is done by assessing both cardiac dynamics and biomarker concentrations to learn the relations between these values. Specifically, the relations between biomarker concentrations and the corresponding values, as well as lagged values, of cardiac dynamics are examined through a network of hidden layers and nodes. As described, the inputs of this model are not limited to two input parameters and the output is not limited to a single biomarker, but can be multivariate in nature.

In an aspect, a method of predicting biomarker concentrations from changes in short-time cardiac dynamics comprises measuring heart rate variability of a subject at rest; measuring heart rate variability of the subject during physical exertion; identifying changes in the heart rate variability at rest verses during physical exertion, and determining an estimated level of a biomarker present in the subject based on the identified changes.

In some embodiments, measuring heart rate variability comprises recording an electrocardiogram of the subject. In more specific embodiments, measuring heart rate variability comprises measuring R-R intervals of the subject. Methods described herein can further comprise estimating biomarker dynamics. Biomarker dynamics can comprise output and secretory dynamics of the biomarker. In some cases, biomarker dynamics comprises output/concentration, pulse amplitude, and/or pulse burst frequency based on patterns in R-R intervals. Methods described herein can further comprise predicting future biomarker concentrations present in the subject based on fluctuations and/or patterns in the R-R interval for a given period of time in some instances. In some instances, the R-R interval range is approximately 600-1200 ms at rest. The given period of time can range from 1-24 hours, and, in some cases, can be more than 24 hours. In a preferred embodiment, the given period of time is at least 8 hours.

In some cases, the biomarker is a regulatory biomarker. Exemplary regulatory biomarker can comprise one or more of growth hormone, glucagon, insulin, nesfatin-1, galanin, cortisol, glucose, insulin-like growth factor 1, or any combination thereof. In some embodiments, the biomarker comprises one or more of glucagon, glucose, insulin, and cortisol.

In some embodiments, the biomarker is hypothalamic-pituitary function-related biomarker. Exemplary hypothalamic-pituitary function biomarker can comprise a pituitary hormone comprising: a growth hormone, somatostatin, growth-hormone releasing hormone, follicle-stimulating hormone, adrenocorticotropic hormone, thyroid-stimulating hormone, luteinizing hormone, vasopressin, oxytocin, and/or gonadotropin-releasing hormone.

The biomarker can be regulated by changes in hypothalamic-pituitary hormones in some instances. In some embodiments, the hypothalamic-pituitary hormones comprise a growth hormone, somatostatin, growth-hormone releasing hormone, follicle-stimulating hormone, adrenocorticotropic hormone, thyroid-stimulating hormone, luteinizing hormone, vasopressin, oxytocin, and/or gonadotropin-releasing hormone.

In methods described herein, an estimated level of the biomarker is a blood or salivary concentration of the biomarker in the subject.

In some embodiments, examining changes in the heart rate variability comprises examining cardiac regulatory dynamics comprising time-domain, frequency-domain, and/or complexity (nonlinear dynamics) of heart rate variability.

The following Examples are exemplary of methods described herein, and should not be considered limiting unless expressly stated.

### Example 1

### General Procedures

Healthy adult males (n=8) were recruited to participate in this study. Each participant reported to the laboratory for a screening- and profile-visit for two phases of this study. Demographic information, exercise training history, body composition, and maximal oxy-gen uptake (VO2max) were assessed on each subject during the screening-visits. The profile-visits consisted of an overnight visit to the laboratory, where serum was collected every 10-minutes, saliva every 2-hrs, and RR-intervals were collected continuously for a 24-hr period. All individuals were healthy adult males who participated in regular moderate-vigorous exercise and were free of any known metabolic, cardiovascular, or pulmonary disease, and with a body composition <18% fat.

An intravenous catheter was placed in either the radial vein or antecubital space of the participant's desired arm and connected to a normal saline drip with a keep-vein-open (KVO) protocol to maintain line-patency (20-30 ml/hr). Blood was collected in a serum separator tube through the closed system and participants were volume-repleted with the waste and a ˜5 ml bolus of normal saline. Blood samples were allowed to clot for 20-40 minutes and were then spun for 12-minutes at 3000 g. Serum was aliquoted into 1.5 ml storage tubes and frozen at −80° C. until assayed. Saliva Collection and Preparation: Saliva samples were collected every two hours in a saliva collection tube and frozen at −80° C. upon the completion of each 24-hr profile until assayed.

All biological samples were run in duplicate. Growth hormone was assayed using commercially available (Ray Biotech) enzyme-linked immunosorbent assays (ELISAs). Salivary cortisol, and serum samples corresponding to the collection times of these salivary samples, were assayed using commercially available (R&D Systems) ELISAs. Nucleobindin/Nesfatin-1 was assayed using ELISAs produced in-house from commercially available (sheep anti-human) capture antibody and (biotinylated sheep anti-human) detection-antibody.

Heart rate data was analyzed using RHRV. Upon importation, instantaneous HR was calculated, plotted, and the files were date and time-stamped. Automatic filtering of the non-interpolated HR was performed for each individual using adaptive thresholds calculated from set parameters and limited by expected physiologic limits (25-200 beats per minutes). These files were then manually inspected, and any additional erroneous beats were removed and updated. Interpolated HR was calculated using linear interpolation so that spectral analysis could be performed. Power spectral analysis was performed using a wavelet-based analysis. The 24-hr HRV spectrum was aggregated into four frequency bands: an ultralow frequency (0.00-0.003 Hz) band, a very low frequency (0.003-0.04 Hz) band, a low frequency (0.04-0.15 Hz) band, and a high frequency (0.15-0.4 Hz) band—all frequency domain measures were log-transformed for analysis. Time-domain analyses, including the standard deviation of the normal RR-intervals (SDNN_{RR}), the standard deviation of the average of normal RR-intervals from all 5-min segments of a 24-hr recording (SDANN_{RR}), root mean square of successive differences of the normal RR-interval (rMSSD_{RR}), and the triangular interpolation of normal RR-intervals (TINN), were calculated over the entire 24-hr period. Sample entropy (SampEnm) for the entire 24-hr period was calculated by averaging the estimation from each embedding dimension—estimations for each embedding dimension (m=2:9) were determined by averaging the estimations from the specified regression ranges (0.15:1).

Subsequently, each 24-hr HRV profile was separated into 3-minute epochs every 10-min (HRVEP)—corresponding with each of the GH samples—totaling 145 epochs (i.e. epoch-1 10 to 13-min, epoch-2 20 to 23-min, etc.). It should be noted that while the time interval for the different epochs was chosen to be 3 minutes for this example, the invention is not limited to this time interval. Instead, the time interval can be any time interval not inconsistent with the objectives of this disclosure. For example, in some embodiments, the time interval I separated into 1 second, 10 seconds, 30 seconds, 45 seconds, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, 10 min, or more than 10 min. Three-minute epochs were used as a means of retaining as much data as possible—considering that 5-min segments would have equaled half of the time between draws. The timing of these epochs (i.e. beginning at every 10th-min as opposed to splitting the epoch around the 10th-min) was chosen since subjects often remained seated during and following these draws—providing the cleanest segments for analysis. Time-domain (SDNN_{EP} and rMSSD_{EP}), as well as nonlinear-indices (SampEn_{EP}) were calculated for each of these 3-min epochs. Power spectral analysis of HRV_{EP} was not performed. These values were subsequently used to create additional time-series that were used to assess the patterned regulation of cardiac-control throughout the day.

Serum cortisol was analyzed every hour for each 24-hr period while salivary cortisol was sampled every 2-hrs for each 24-hr period. Fit-validation was performed on both serum and salivary cortisol samples by fitting each individual profile from 1-10 polynomials and comparing model fit across conditions and individuals. The R2, adjusted R2, and mean absolute error (MAE) of each ordered-model were plotted and compared on an individual and profile basis. Third- and fourth-order polynomials generally provided the best-fits without any significant improvements in model fit—the final model used to fit the data was determined by comparing the improvement in fit relative to each increase in polynomial order and by comparing the autocorrelation of the residual errors from these two models. The inherent nature of these models, which were used to impute values at minutes 10, 20, 30, 40, and 50 of each hour, is that they are individualized and follow a robust circadian pattern. The variability of these imputations was based on the variance of the models fit to the raw data (24 time-points—one sample every hour for 24-hr).

Area under the curve (AUC) is a commonly reported value in endocrine research. Many methods, such as the trapezoidal rule, polynomial interpolation of various degrees, Simpson's integration, and cubic interpolatory splines, have been proposed for calculating the AUC. The trapezoidal rule estimates the integral of the line by dividing each pro-file into multiple sections of equal length and subsequently estimating each segment by taking the integrand of a constant value whereas the cubic spline method calculates the area under the natural cubic spline interpolation. The cubic spline and the trapezoidal methods were both used to calculate a subset of all AUC values. These data (not presented) were not statistically different from one-another, as has been previously reported, and thus the trapezoidal method was used for computational efficiency.

Mean hourly output was calculated for specific periods of time throughout the 24-hr period; all of which were calculated as output per hour. Values were calculated over the course of the entire 24-hr sampling period as well as daytime (corresponding to the two hours following the onset of exercise, 10:00 am-12:00 pm) and nighttime hours (11:00 pm-6:00 am).

### Example 2

### Relationship Between Hypothalamic-Pituitary Regulation and Cardiac Control at-Rest

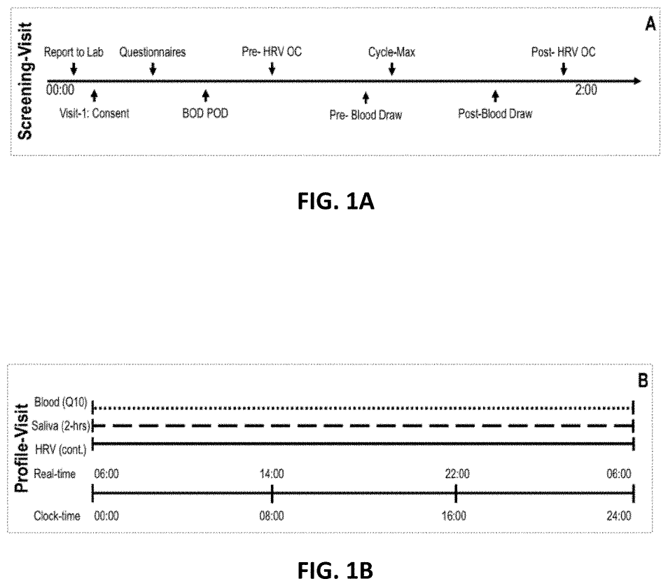

Quantification of the dynamic relationships between markers of hypothalamic-pituitary regulation and cardiac control at rest were determined to establish a baseline understanding of the dynamics of these nodal biomarkers while at rest. Specifically, GH, cortisol, and HRV were assessed in healthy males during a 24-hr period of rest and with the assumption that assessing the dynamics of these time-series would provide context to the regulatory relationships among these systems and evidence for a common attractor (or, a point at which these systems evolve towards) between these markers. FIGS. 1A and 1B show the at-rest study design, with FIG. 1A showing the screening visit of a participant, and FIG. 1 B showing the profile visit of the participant where blood was sample from an IV catheter at the given intervals and RR-recording was sampled continuously.

Using the procedures described generally in Example 1, all data management and statistical procedures were performed using R Statistics version 3.5.0 (177). For specific information pertaining to data cleaning, the handing of missing data, and the calculations of the following dependent variables, please see Chapter III—General methods and data processing. Twenty-four-hour heart rate variability (HRV) indices included the standard deviation of the normal RR-intervals (SDNN_{RR}), the standard deviation of the average of normal RR-intervals from all 5-min segments of a 24-hr recording (SDANN_{RR}), the root mean square of successive differences of the normal RR-interval (rMSSD_{RR}), and the triangular interpolation of nor-mal RR-intervals (TINN_{RR}) and sample entropy (SampEn_{RR}).

Each of these 24-hr HRV profiles were processed into 3-min epochs, taken every 10-min (i.e. 10-13-min, 20-23-min, 30-33-min). As previously stated, it should be noted that while the time interval for the different epochs was chosen to be 3 minutes for this example, the invention is not limited to this time interval. Instead, the time interval can be any time interval not inconsistent with the objectives of this disclosure. For example, in some embodiments, the time interval I separated into 1 second, 10 seconds, 30 seconds, 45 seconds, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, 10 min, or more than 10 min. The variability and complexity of each of these short-time segments were assessed and used to create additional time-series (HRVEP) with values corresponding to the timing of the serum samples. The dynamics of these HRV_{EP} profiles were assessed to better understand the patterned regulation of cardiac control throughout the day (i.e. how the variability and complexity of 3-min RR-recordings changed every 10-min throughout the 24-hour period). SampEn and recurrence quantification analysis (RQA) were used to assess the dynamics of the univariate time-series. RQA indices included (REC), determinism (DET), ratio of DET/REC (RA-TIO), and the length of the longest diagonal line (Lmax). Cross-RQA (cRQA) was used to examine the dynamics between bivariate time-series. These comparisons included GH-SDNN_{EP}, GH-rMSSD_{EP}, GH-SampEn_{EP}, GH-cortisol, cortisol-SDNN_{EP}, cortisol-rMSSD_{EP}, cortisol-SampEn_{EP}, SDNN_{EP}-Sampen_{EP}, and rMSSD_{EP}-SampEn_{EP}. The REC, DET, Lmax, entropy, and normalized-entropy (entropyNL: normalized for the number of lines) were calculated for each analysis. The joint-Shannon-entropy, and mutual information between the bivariate time-series, was calculated for GH-SDNN_{EP}, GH-rMSSD_{EP}, and GH-SampEn_{EP}.

The autocorrelation function (ACF) and average mutual information (AMI) for the GH and HRV_{EP} data were both calculated for each respective time-series. The time-lag calculated from AMI was used to calculate the embedding dimension for each time-series and the embedding dimension was subsequently used to calculate the Takens' vector (used to reconstruct the state space of each time-series) and the correlation dimension (a measure of fractal dimension). SampEn and RQA were calculated using the embedding dimension m and radius r optimized for each time-series. Parameters used to calculate cRQA were standardized across all profiles (m=3, delay=7, r=100); all time-series were rescaled for cRQA analysis. Joint-entropy and the mutual information were also calculated using also calculated using standardized parameters across all measures (bins=10).

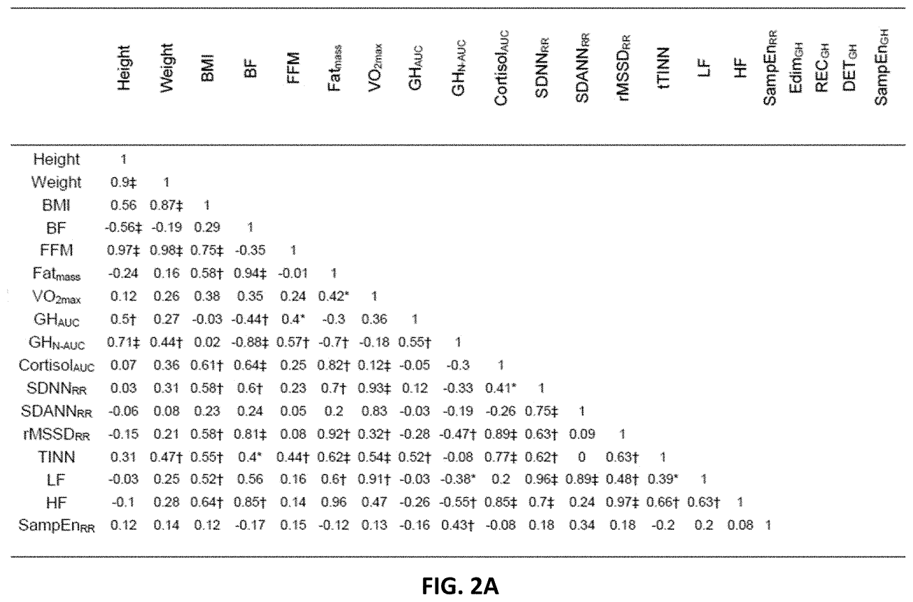

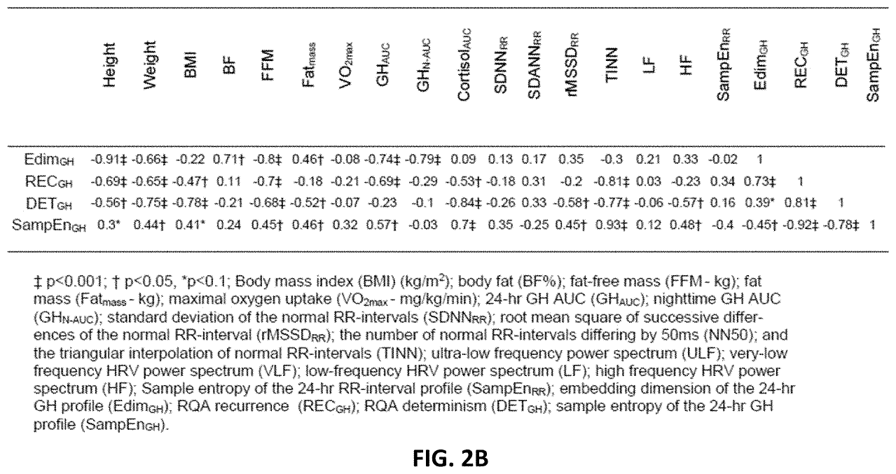

Subject demographics are presented in Table 1. HRV indices from the entire 24-hr period are presented in Table 2 while measures of GH and cortisol output are presented in Table 3. Correlations among demographic information, characteristics of biomarker output, as well as the variability and complexity indices from the 24-hr HRV indices and HRV_{EP} time-series are presented in the tables shown in FIGS. 2A and 2B. In general, HRV_{RR} indices were associated with GHN-AUC but not GHAUC, while the dimensionality and dynamics (i.e. ACF, AMI, Edim, REC, DET, RATIO, Lmax, and SampEn) of GH output were significantly associated with GHAUC but not GHN-AUC. No statistical differences were observed between the joint-entropy measures (F_{(2.10)}=0.28, p=0.76). The nonlinear parameters and indices from the HRV_{EP} data are presented in Table 4. Joint-entropy among GH and SDNN_{EP}, rMSSD_{EP}, or SampEn_{EP} are provided in Table 5. Table 6 provides information on cRQA; REC, DET, Lmax, entropy, and entropyNL for GH-SDNN_{EP}, GH-rMSSD_{EP}, GH-SampEn_{EP}, GH-cortisol, cortisol-SDNN_{EP}, cortisol-rMSSD_{EP}, cortisol-SampEn_{EP}, SDNN_{EP}-Sampen_{EP}, and rMSSD_{EP}-SampEn_{EP}.

TABLE 1
Subject Demographics

| | | |
|---|---|---|
| Age | 25.7 | ±2.4 |
| Height (cm) | 174.7 | ±7.8 |
| Weight (kg) | 69.8 | ±12.1 |
| BMI (kg/m²) | 22.7 | ±2.5 |
| BF (%) | 9.0 | ±2.7 |
| FFM (kg) | 64.6 | ±10.6 |
| Fatₘₐₛₛ (kg) | 6.3 | ±2.0 |
| VO₂ₘₐₓ (ml/kg/min) | 67.8 | ±9.0 |

(n = 7) Data presented as mean ± sd

Body mass index (BMI); Body fat (BF); fat-free-mass (FFM); fat-mass (Fatₘₐₛₛ); maximal oxygen uptake (VO₂ₘₐₓ).

TABLE 2
HRV Indices Over 24-hr of Rest in Young Healthy Males

| | | |
|---|---|---|
| SDNN_{RR} | 197.7 | ±43.1 |
| rMSSD_{RR} | 82.9 | ±37.8 |
| TINN | 847.1 | ±223.7 |
| ULF | 13.2 | ±1.1 |
| VLF | 13.3 | ±1.1 |
| LF | 13.3 | ±1.1 |
| HF | 8.0 | ±1.7 |
| SampEn_{RR} | 1.6 | ±0.2 |

(n = 7) Data presented as mean ± sd

Standard deviation of the normal RR-intervals (SDNN_{RR}); root mean square of successive differences of the normal RR-interval (rMSSD_{RR}); triangular interpolation of normal RR-intervals (TINN); ultra-low frequency power spectrum (ULF); very-low frequency HRV power spectrum (VLF); low-frequency HRV power spectrum (LF); high frequency HRV power spectrum (HF); Sample entropy of the 24-hr RR-interval profile (SampEn_{RR}).

TABLE 3
GH and Cortisol Output During Rest in Young Healthy Males

| | GH | Cortisol |
|---|---|---|
| | ng/ml | μg/dl |
| AUC₂₄ | 1060.03 ± 151.27 | 1141.03 ± 264.10 |
| AUC_{N} | 608.03 ± 84.33 | 319.76 ± 91.21 |
| Peak_{N} | 5.54 ± 0.33 | |
| Nadir | 0.09 ± 0.03 | |

(n = 7) Data are presented
as mean ± se

24-hr area under the curve (AUC) (AUC₂₄); nighttime AUC (AUC_{N}); highest observes concentration-corresponding to nighttime hours (Peak_{N}); lowest observed concentration during the 24-hrs (Nadir)

TABLE 4
Nonlinear Parameters of GH and HRV_{EP} Profiles

| | ACF | AMI | m | REC | DET | RATIO | Lₘₐₓ | SampEn |
|---|---|---|---|---|---|---|---|---|
| GH | 7.1 ± 1.1 | 5.9 ± 1.8 | 9.7 ± 1.3 | 0.15 ± 0.14 | 0.89 ± 0.12 | 12.40 ± 11.31 | 32.71 ± 20.25 | 0.46 ± 0.17 |
| SDNN_{EP} | 4.3 ± 7.4 | 1.0 ± 0.0 | 8.1 ± 1.8 | 0.19 ± 0.13 | 0.96 ± 0.04 | 11.27 ± 12.12 | 34.86 ± 21.84 | 0.61 ± 0.11 |
| rMSSD_{EP} | 10.1 ± 9.1 | 1.3 ± 0.5 | 6.4 ± 1.1 | 0.34 ± 0.30 | 0.88 ± 0.16 | 5.43 ± 6.10 | 58.43 ± 42.58 | 0.59 ± 0.17 |
| SampEn_{EP} | 1.0 | 1.0 | 8.0 ± 0.8 | 0.36 ± 0.08 | 0.98 ± 0.005 | 2.89 ± 0.80 | 49.71 ± 9.96 | 0.77 ± 0.11 |

(n = 7) Data presented as mean ± sd
The mean ± sd autocorrelation function (ACF), average mutual information (AMI), optimal-embedding dimension (m), recurrence (REC), determinism (DET), ratio of DET/REC (RATIO), length of the longest diagonal line (Lₘₐₓ), and sample entropy (SampEn) values for the 24-hr GH profile and the HRV_{EP} data; standard deviation of the normal RR-intervals (SDNN_{EP}); root mean square of successive differences of the normal RR-interval (rMSSD_{EP}) and SampEn_{EP}.

TABLE 5
Joint-Entropy and Mutual Information for GH-HRV_{EP} Profiles

| Joint entropy | | | Mutual information | | |
|---|---|---|---|---|---|
| GH- | | | GH- | | |
| SDNN_{EP} | rMSSD_{EP} | SampEn_{EP} | SDNN_{EP} | rMSSD_{EP} | SampEn_{EP} |
| 2.66 ± 0.47 | 2.69 ± 0.29 | 2.79 ± 0.24 | 0.23 ± 0.06 | 0.27 ± 0.08 | 0.20 ± 0.03 |

(n = 7) Data presented as mean ± sd
Joint-entropy and mutual information calculations were performed on joint time-series of GH and HRV_{EP} profiles. HRV indices included: Standard deviation of the normal RR-interval (SDNN_{EP}), the root mean square of successive differences (rMSSD_{EP}), and sample entropy (SampEN_{EP})

TABLE 6
CRQA Analysis of Biomarker and HRV_{EP} Profiles During Rest

| | REC | DET | Lₘₐₓ | Entropy | Entropy_{NL} |
|---|---|---|---|---|---|
| GH-SDNN_{EP} | 56.25 ± 8.85 | 85.79 ± 8.05 | 21.14 ± 17.24 | 1.69 ± 0.36 | 0.68 ± 0.15 |
| GH-rMSSD_{EP} | 60.19 ± 8.04 | 88.87 ± 12.49 | 41.86 ± 24.36 | 2.03 ± 0.46 | 0.60 ± 0.08 |
| GH-SampEn_{EP} | 71.75 ± 10.21 | 98.63 ± 1.64 | 74.71 ± 29.18 | 3.13 ± 0.46 | 0.75 ± 0.03 |
| GH-Cortisol | 47.86 ± 6.14 | 87.77 ± 10.94 | 10.14 ± 1.46 | 1.28 ± 0.31 | 0.62 ± 0.16 |
| Cortisol-SDNN_{EP} | 56.69 ± 8.43 | 82.70 ± 8.99 | 9.14 ± 2.54 | 1.64 ± 0.30 | 0.81 ± 0.09 |
| Cortisol-rMSSD_{EP} | 60.26 ± 8.07 | 87.35 ± 12.64 | 10.29 ± 1.60 | 1.62 ± 0.31 | 0.74 ± 0.10 |
| Cortisol-SampEn_{EP} | 45.50 ± 9.76 | 85.15 ± 16.83 | 4.57 ± 1.62 | 0.56 ± 0.31 | 0.53 ± 0.31 |
| SDNN_{EP}-SampEn_{EP} | 56.05 ± 8.98 | 85.44 ± 8.37 | 19 ± 18.10 | 1.65 ± 0.36 | 0.71 ± 0.17 |
| rMSSD_{EP}-SampEN_{EP} | 59.89 ± 8.39 | 88.27 ± 13.31 | 37.14 ± 27.55 | 1.87 ± 0.51 | 0.62 ± 0.13 |

(n = 7) Data presented as mean ± sd
Growth hormone (GH); Variability and complexity indices calculated on the HRV_{EP} profiles: Quantification measures from cRQA: recurrence (REC), determinism (DET); maximal line length (Lₘₐₓ); Shannon-entropy of the lines longer than the minimum line length (Entropy), Entropy normalized to the number of lines (Entropy_{NL}).

To summarize, HRV indices were assessed in two forms—a continuous 24-hr profile and an epoched-profile which consisted of 3-min windows analyzed every 10-min throughout the 24-hr window (totaling 145 unique samples). The indices of 24-hr HRV for this study are consistent with established norms. Furthermore, the high correlations among LF power and SDNN as well as HF power and rMSSD in our study are congruent with well-established observations.

HRV_{EP} was used to assess the patterned change in cardiac control throughout the day. An immediate comparison between the 24-hr indices of HRV and the means of HRV_{EP} data would be inappropriate given the different lengths of the time-series, the different physiological representations of these measures for each respective time-series, and the limitations associated with mathematical calculations being derived from time-series of such drastically different lengths. However, optimal time-lag (calculated from AMI) and the optimal embedding dimension for each of the HRV_{EP} time-series were similar across profiles (SDNN_{EP} lag=1, m=8.1; rMSSD_{EP} lag=1.3, m=6.4; SampEn_{EP} lag=1, m=8). While the common standard within the HRV literature uses a set embedding dimension for the analysis of entropy measures for raw RR-recordings, the optimal embedding dimensions for raw RR-recordings (at least from our observations) seem to vary significantly. While not intending to be bound by theory, this suggests that these HRV_{EP} profiles may be a more robust method of assessing cardiac control throughout the day—keeping in mind that each short-time RR-recording can be impacted by acute physical, mental, or psychophysiological stresses.

In a normal, healthy, RR-recording, both SDNN_{EP} and rMSSD_{EP} follow the same trend as the raw RR-intervals; and a relatively stable RR-recording is observed throughout the day with an upward, positive, trend during the nighttime hours. However, SampEn_{EP} was stationary throughout the entire 24-hr period. These observations suggest that while the variability measures (SDNN_{EP} and rMSSD_{EP}) are impacted by raw changes in the RR-interval throughout the course of the 24-hr period, the complexity (SampEn_{EP}) of these profiles is more robust against raw changes related to environmental, behavioral, and circadian conditions/factors.

The dynamics (REC, DET, RATIO, Lmax, and SampEn) of these HRV_{EP} profiles were notably different for each time-series, which suggests that the dynamics of the regulatory patterns of cardiac control throughout the day are different between individuals whereas the HRV_{RR} indices were rather homogeneous.

Both the GH and cortisol profiles were similar to what has been previously reported within the literature. Mean GH_{AUC}, GH_{N-AUC}, and GH_{N-Peak} were similar to values previously reported in heathy, young adult males. Similarly, the relations among these markers were consistent with previous literature; GH_{AUC} was correlated with GH_{N-AUC}. In addition, mean cortisol levels were consistent with previous observations and followed a circadian pattern with higher concentrations during the morning hours and lower concentrations during the nighttime hours.

Logistically, these analyses were broken into three key components. Firstly, the correlations among the demographic, hormonal output, HRV_{RR}, and dynamics associated with the HRV_{EP} profiles are presented in the tables shown in FIGS. 2A and 2B. While the overarching argument is that these are dynamic systems, consideration of some of the more-straightforward linear relations (i.e. correlations) among mean measures from time-series of each of these variables was warranted. These comparisons were made to provide preliminary evidence surrounding the relations between indices of cardiac control, hormonal output (GH and cortisol), and the dynamics of GH output.

Interestingly, GH_{AUC} was significantly correlated with TINN, which is a geometric index of HRV (the integral of the density distribution) but not with other HRV_{RR} index. Meanwhile, GH_{N-AUC} was negatively correlated with rMSSD_{RR} and HF power but positively correlated with SampEn_{RR}. Cortisol_{AUC} was also positively correlated with rMSSD_{RR}, TINN, and HF power. The optimal embedding dimension for the GH profile was strongly negatively associated with both GH_{AUC} and GH_{N-AUC} and positively correlated with REC_{GH}. Simplistically, this suggests that the time-series with more dimensionality revisited the same dimensional space more often than others. While REC_{GH} having a high positive correlation with DET_{GH} and strong negative correlation with SampEn_{GH} may not be extremely surprising, the negative correlations between DET_{GH} with HF power, TINN, and rMSSD_{RR} suggest a unique relationship between the dynamics of the hypothalamic-pituitary axis and cardiac control (specifically, parasympathetic regulation of cardiac control). In addition, SampEn_{GH} was positively associated with both rMSSD_{RR}, TINN, and HF power and together, these findings begin to suggest that there may be higher-order regulation between parasympathetic regulation of cardiac control and hypo-thalamic-pituitary regulation. This higher-order regulation may be representative of a common attractor between these systems; an attractor being a point in which systems evolve toward.

Secondly, comparison of the dimensionality and dynamics of the GH profiles with the dynamics of the HRV_{EP} profiles was also warranted. Interestingly, the variability in the ACF of SDNN_{EP} and rMSSD_{EP} profiles were notably higher than the variability observed within the GH profiles. However, controlling for the lagged time-series produced much lower, and less variable, estimates through AMI. Nevertheless, the discrepancy between these indices, compared to those of GH and SampEn_{GH} for example, suggest that these pro-files (SDNN_{EP} and rMSSD_{EP}) retain a significant amount of information about the system from one observation to the other. The optimal embedding dimension seemed to be notably higher in the GH profiles compared to any of the HRV_{EP} profiles. The optimal time-lag, assessed by both ACF and AMI, was calculated at 1 for all of the profiles with a rather invariant optimal embedding dimension of 8. While the REC, DET, RATIO, Lmax and SampEn of each of these time-series provide interesting context to the individuality of the dynamics associated with GH output, the significant takeaways from these analyses are those relating to the dynamics of these profiles.

The component of these analyses provided additional context to these observations by assessing the dimensionality between combinations of these time-series. Specifically, joint-entropy and mutual information were calculated to assess the orderliness of the entire length of these time-series and how much information is shared among them, respectively. These measures failed to provide any significant context to the findings previously discussed, however, this study design was not designed to provide adequate power to make this comparison and future studies should further investigate the mean differences in joint-entropy between GH-SDNN_{EP}, GH-rMSSD_{EP}, and GH-SampEn_{EP}.

Whereas joint-entropy assesses the probability of any two values occurring together, cRQA assesses the dynamical behavior of two time-series within the same phase-space. Quantification of these recurrence plots further supports the previous conclusion that the dynamics between these profiles are certainly measurement-specific, but may also be individual-specific in some cases. For instance, there appear to be distinct differences between the REC and DET of specific crossed-profiles. The REC and DET of GH-SDNN_{EP} and GH-rMSSD_{EP} were notably lower than GH-SampEn_{EP} while the entropy of the former two measures was also relatively (similar and) lower than that of GH-SampEn_{EP}. Nevertheless, the dynamics between cortisol-SDNN_{EP} and cortisol-rMSSD_{EP} were relatively comparable to those of GH-SDNN_{EP} and GH-rMSSD_{EP}. Interestingly, cortisol-SampEn_{EP} was not only lower than measurements of GH-SampEn_{EP}, but lower than both cortisol-SDNN_{EP} and cortisol-rMSSD_{EP}. While the physiologic consequences of this remain to be elucidated, it further suggests that there is a common attractor between these systems. While this attractor may be more clearly represented in one measure (of cardiac control, assessed through HRV_{EP}) compared to another, these relationships need to be better understood by assessing how these measures change following various perturbations.

Nevertheless, some of the consistency observed between HRV_{EP} (specifically SDNN_{EP} and rMSSD_{EP}) with GH and cortisol may have, at least in part, to do with the SCN and its regulation of various physiologic rhythms. Circadian control across physio-logic systems occurs through the SCN where light from the optic nerve stimulates the SCN via the retinohypothalamic tract (RHT) and entrains the SCN to the light-dark cycle. Within a healthy system, the SCN controls peripheral clocks and dysregulation within the SCN can result in peripheral clocks becoming further dysynchronized. The integration and coordination between central and peripheral clocks regulate the rhythmic control of circadian glucose concentrations and other physiologic regulatory mechanisms within the liver, pancreas, skeletal muscle, intestine, and adipose tissue.

In summary, although the relationships between hypothalamic-pituitary and cardiac regulation appear to be highly individual, this data suggests that they share a common attractor within the hierarchy of physiologic regulation. These findings provide important context to the overall regulatory organization of the physiologic system and further elucidating these relationships in healthy and diseased systems at rest and following a perturbation can provide important context to the manifestation and progression of disease. Furthermore, better understanding the regulatory dynamics of the hypo-thalamic-pituitary axis and cardiac control following a perturbation to the system will provide additional context to the findings outlined here and help inform decisions about how changes in a specific index, or indices, of HRV_{EP} best represent changes in hypothalamic-pituitary control.

### Example 3

### Relationship Between Hypothalamic-Pituitary Regulation and Cardiac Control after Exercise

Delineation of specific exercise-induced changes in the dynamic relationships between measures of hypothalamic-pituitary regulation and cardiac control compared to rest in healthy males was determined. Healthy adult males (n=7) reported to the laboratory for two 24-hr profiles where serum samples were collected Q10 and RR-intervals were collected continuously using the general methods described in Example 1. Rest and exercise conditions were randomly assigned to these profile-visits; the exercise conditions consisted of 5 Wingate bouts with 3-min recovery between each. Measures of variability and complexity used to analyze the 24-hr HRV profile included high frequency power (HF), the standard deviation of the normal RR-intervals (SDNN_{RR}), the root mean square of successive differences of the normal RR-interval (rMSSD_{RR}), and sample entropy (SampEn_{RR}). Additional time-series were created from these 24-hr recordings by epoching these time-series (HRV_{EP}); the variability (SDNN_{EP} and rMSSD_{EP}) and complexity (SampEn_{EP}) of each 3-min epoch, taken every 10-min (i.e. 10 to 13-min, 20 to 23-min, etc.) throughout the 24-hr period, was assessed and that value used to quantify the dynamic patterns in the variability and complexity of cardiac control throughout the day. Specifically, these time-series were used to assess changes in cardiac control throughout the 24-hr period univariately and in conjunction with GH output at rest and following a high-intensity exercise perturbation. The dynamics of these profiles were assessed using recurrence analysis (RQA) and SampEn.

As described herein, multivariate analysis of variance (MANOVA) indicated a significant difference (p=0.04) in the optimal parameters chosen to analyze the dynamics of each profile between exercise and resting conditions. There was no difference in the recurrence (REC) of GH, however, the determinism (DET) of the GH profile interacted with changes in fitness between conditions (p=0.04).

The findings related to exercise-induced changes in variability and complexity suggest a common attractor among the hypothalamic-pituitary axis and cardiac control; assessed by GH and HRV_{EP} respectively. Assessing the relations among these profiles in parallel can in some cases provide a method of creating a scalable model that can predict GH output from changes in HRV_{EP} profiles.

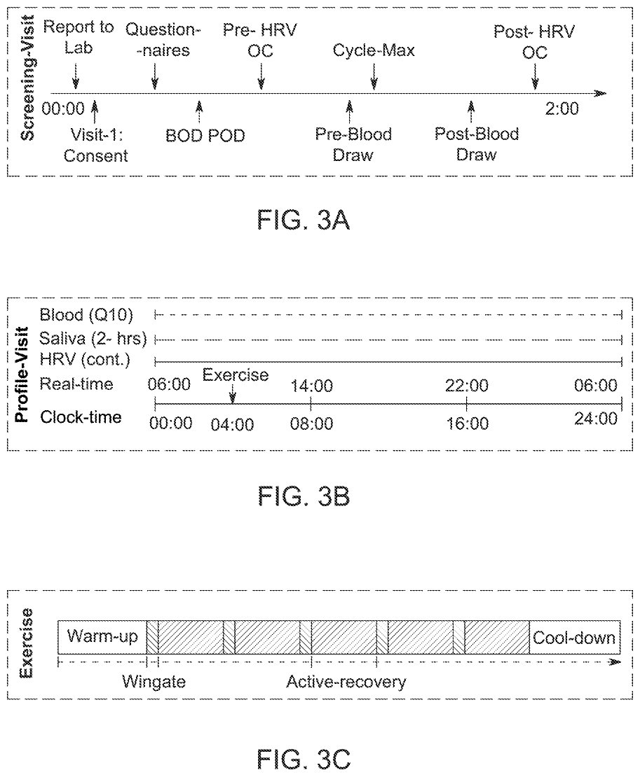

FIGS. 3A-3C show an exercise study design, with FIG. 3A showing a screening visit of a participant; FIG. 3B showing the profile visit of the participant where blood was sample from an IV catheter at the given intervals, saliva collection was performed at given intervals, and RR-recording was sampled continuously; and FIG. 3C shows the exercise protocol that was completed on a cycle ergometer.

During the screening-visit (FIG. 3A), each participant provided training history. Body composition was assessed with COSMED's BOD POD. Participants then completed a ramp test (100 W+25 W/min) on the cycle ergometer to volitional fatigue (Lode Excaliber Sport). Breath-by-breath oxygen uptake was collected (ParvoMedics TrueOne 2400).

The profile-visit (FIG. 3B) was completed no less than 48-hrs and no more than 2-weeks following the screening-visit. Participants reported to the laboratory to complete a 24-hr profile beginning at 6 AM (00:00). An intravenous catheter was placed in either the radial vein and antecubital space and serum samples (3 ml) were collected every 10-minutes (Q10)-totaling 145 samples. Normal RR-intervals were collected via Polar HR monitor (V800). During the 24-hr sampling period, subjects were allowed to ambulate throughout the day. Participants were restricted to water between the hours of 8 AM-10:30 AM (02:00-04:30) to standardize macronutrient intake prior to the exercise bout. Individuals ate breakfast ˜7:30 AM (01:30), lunch ˜1:00 PM (07:00), and dinner ˜8:00 PM (14:00). All food and beverages consumed by the participants were detailed in a dietary log and participants were asked to consume foods of similar macronutrient composition during the second profile-visit. Participants were permitted to go to bed at their discretion, with a mandatory lights-out policy at 11:00 PM (17:00). Blood sample collection and analysis were performed as described in Examples 1 and 2.

For the exercise protocol, following a warm up at a self-selected workload (<50 watts) for a period of 5-minutes, participants began the high-intensity-exercise session. Participants completed five 30-second all out Wingate bouts on the cycle ergometer with a force equal to 0.075*body weight (kg) applied to the flywheel. Each of the five 30-sec-ond bouts was separated by a 3-minute active recovery period on the cycle ergometer.

GH was assayed using commercially available enzyme-linked immunosorbent assays (ELISAs) (Ray Biotech). Serum cortisol was assayed every hour using commercially available ELISAs (R&D Systems). Area under the curve (AUC) was calculated for GH during the entire 24-hr period (GH_{AUC}), the nighttime (11:00 pm-6:00 am) (GH_{N-AUC}), and daytime (10:00 am-12:00 pm) (GHEX)—corresponding to the timing of the exercise bout—hours to assess changes in GH output while the secretory rate was calculated each of these timeframes per hour. Total cortisol output was calculated for the entire 24-hr period (cortisolAUC), nighttime cortisol (cortisolN-AUC), and daytime (cortisolD-AUC)—all times used for these calculations matched those used to calculate the GH output. Peak (nighttime) GH, peak GH following the exercise bout (PeakEX), and nadir concentrations were also calculated for both exercise and resting conditions.

Data was assessed in the same manner as described in Examples 1 and 2.

Participant demographics for each condition are presented in Table 7. The average body weight, BF, and VO₂ₘₐₓ were slightly higher in the exercise condition compared to rest, however, none of these differences reached significance.

TABLE 7
Participant Demographics During Exercise and Resting Conditions

| | Exercise | | Rest | |
|---|---|---|---|---|
| Age | 25.4 | ±2.6 | 25.7 | ±2.4 |
| Height (cm) | 174.7 | ±7.8 | 174.7 | ±7.8 |
| Weight (kg) | 71.2 | ±10.8 | 69.8 | ±12.1 |
| BF (%) | 9.8 | ±3.3 | 9.0 | ±2.7 |
| FFM (kg) | 64.2 | ±10.0 | 64.6 | ±10.6 |
| VO₂ₘₐₓ (ml/kg/min) | 71.2 | ±11.2 | 67.8 | ±9.0 |

(n = 7) Data presented as mean ± sd

Exercise and resting conditions were separated by a minimum of 8-weeks. No statistical differences were observed between these measures. Body fat (BF), fat-mass (Fatₘₐₛₛ); and maximal oxygen uptake (VO₂ₘₐₓ)

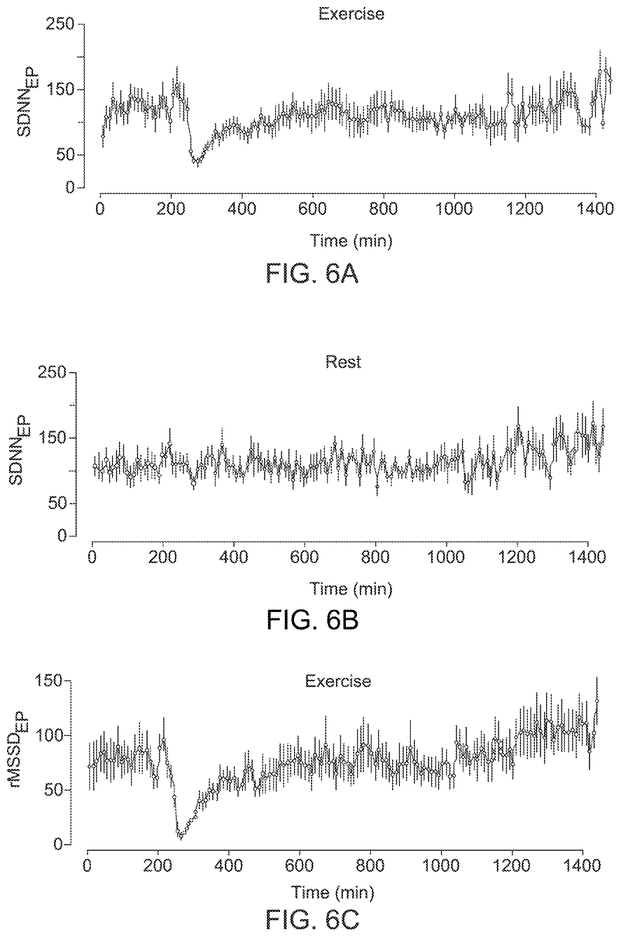

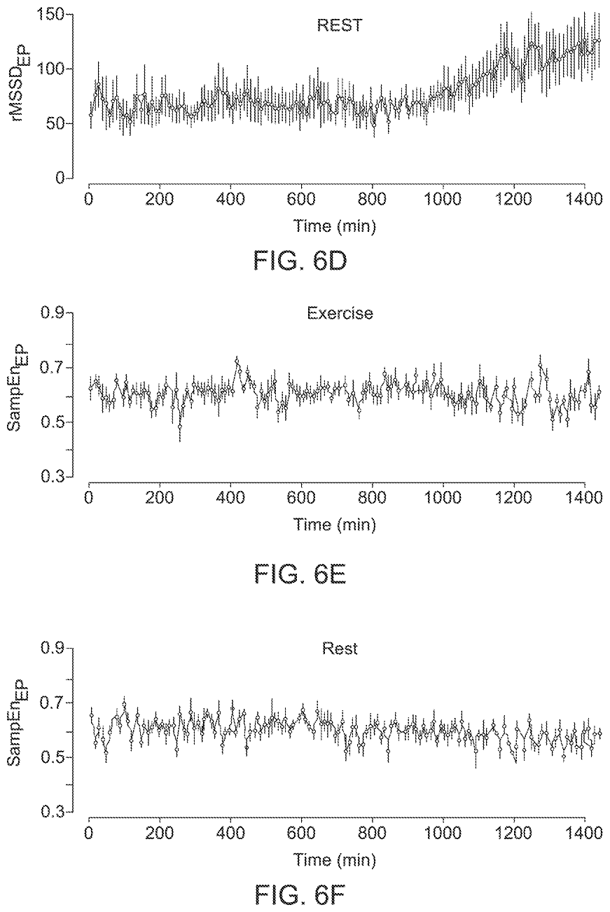

HRV indices from the entire 24-hr exercise and resting profiles are presented in Table 8. The multivariate model testing differences between 24-hr HRV indices indicated no significant difference between conditions (V=0.35, F_{(1.6)}=3.29, p=0.12), how-ever, the exploratory univariate tests indicated some interesting findings. A near significant difference in LF power (F_{(1.6)}=4.32, p=0.08) and a significant difference in SDNN_{RR} between exercise and resting-conditions (F_{(1.6)}=12.28, p=0.04) was observed. SampEn_{RR} was significantly different between conditions (F_{(1.6)}=78.65, p<0.001). No differences were observed for cortisol output measures between conditions (V=0.05, F_{(1,3)}=0.32, p=0.59). The mean Exercise and Rest, respectively, of SDNN_{EP}, rMSSD_{EP}, and SampEn_{EP} profiles are presented in FIGS. 6A-6F, where SDNN_{EP} (FIGS. 6A, 6B), rMSSD_{EP} (FIGS. 6C, 6D), SampEn_{EP} (FIGS. 6E, 6F), and cortisol (FIGS. 6G, 6H), for exercise and resting profiles.

TABLE 8
24-hour HRV Indices During the Exercise and Resting Conditions.

| | |
|---|---|
| Exercise | Rest |

| | | | | |
|---|---|---|---|---|
| SDNN_{RR} | 216.0 | ±43.7 † | 197.7 | ±43.1 |
| SDANN_{RR} | 162.4 | ±30.7 | 152.3 | ±40.3 |
| rMSSD_{RR} | 79.7 | ±38.0 | 82.9 | ±37.8 |
| TINN | 978.5 | ±278.1 | 847.1 | ±223.7 |
| LF | 13.6 | ±0.7 * | 13.3 | ±1.1 |
| HF | 8.0 | ±1.7 | 8.0 | ±1.7 |
| SampEn_{RR} | 0.7 | ±0.1 ‡ | 1.6 | ±0.2 |

(n = 7) Data presented as mean ± sd

‡ p < 0.001;
† p < 0.05,
* p < 0.1.
Standard deviation of the normal RR-interval (SDNN); root mean square of successive differences of the normal RR-interval (rMSSD); the number of normal RR-intervals differing by 50 ms (NN50); and the triangular interpolation of normal RR-intervals (TINN); ultra-low frequency power spectrum (ULF); very-low frequency HRV power spectrum (VLF); low-frequency HRV power spectrum (LF); high frequency HRV power spectrum (HF); Sample entropy of the 24-hr RR-interval profile (SampEn_{RR}).

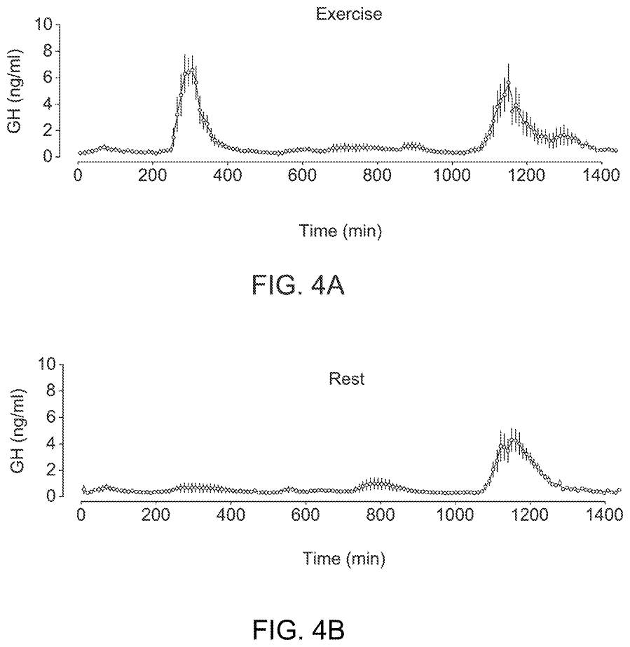

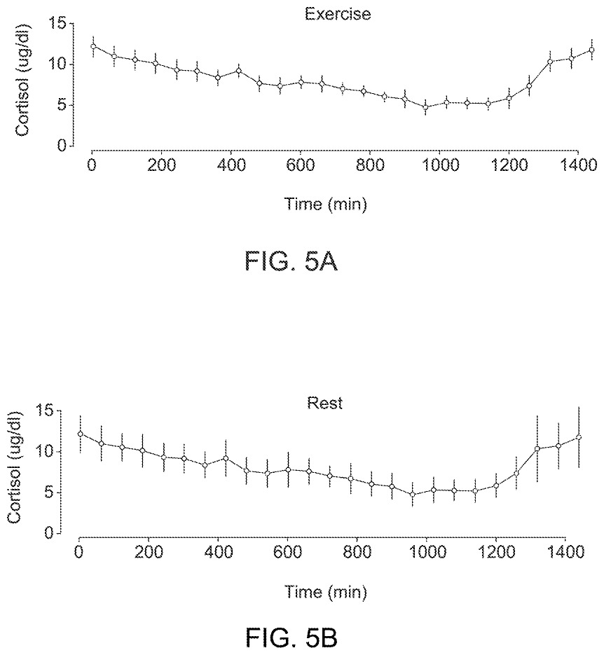

Measures of GH and cortisol output are presented in Table 9. FIGS. 4A-5B present the mean exercise and resting profiles for GH and cortisol respectively. Non-linear parameters and indices from the HRV_{EP} data are presented in Table 10. The MANCOVA testing differences in GH output characteristics indicated a near-significant difference between conditions (0.74, F_{(1,3)}=8.83, p=0.05) and interactions between the change in BF (V=0.82, F_{(1,3)}=13.67, p=0.03) and FFM (V=0.95, F_{(1,3)}=63.26, p=0.005). Univariate follow up tests indicated significant differences in the interactions between changes in BF (F_{(1,3)}=15.43 p=0.03), changes in FFM (F_{(1,3)}=23.7, p=0.002), and changes in VO2max (F_{(1,3)}=9.94, p=0.05) with condition. Additionally, interactions between changes in BF (F_{(1,3)}=8.57 p=0.06), changes in FFM (F_{(1,3)}=12.55, p=0.05), and changes in VO2max (F_{(1,3)}=20.40, p=0.02) with condition were observed for GH_{N-Peak}. The exercise bout resulted in a significant GH response (F_{(1,3)}=34.45, p<0.005) but GHNadir was not different between conditions (F_{(1,3)}=0.37, p=0.58). No significant differences in cortisol output were observed were observed between conditions (V=0.06, F_{(1.6)}=0.32, p=0.59).

TABLE 9
GH and Cortisol Output During the Exercise and Resting Conditions.

| | Exercise | | Rest | |
|---|---|---|---|---|
| | GH | Cortisol | GH | Cortisol |
| | ng/ml | μg/dl | ng/ml | μg/dl |
| AUC₂₄ | 1602.21† ± 264.27 | 986.16 ± 116.75 | 1080.03† ± 151.27 | 1141.03 ± 264.10 |
| AUC_{N} | 702.41† ± 162.35 | 271.59 ± 39.30 | 608.03† ± 84.33 | 319.76 ± 91.21 |
| AUC_{EX} | 458.14† ± 90.69 | 107.42 ± 14.87 | 73.06† ± 39.44 | 108.61 ± 20.00 |
| Peak_{N} | 6.57† ± 1.27 | | 5.54 ± 0.86 | |
| Peak_{EX} | 7.98† ± 1.46 | | 1.57† ± 0.33 | |
| Nadir | 0.09 ± 0.03 | | 0.09 ± 0.03 | |

(n = 7) Data are presented as mean ± se
‡ p < 0.001,
†p < 0.05.
24-hr area under the curve (AUC₂₄); nighttime AUC (AUC_{N}); AUC during exercise hours-corresponds to 10:00am-12:00am/04:00-06:00 clock-time (AUC_{EX}); peak nighttime concentration (Peak_{N}); peak during post exercise hours-corresponds to 10:00am-12:00pm/04:00-06:00 clock-time (Peak_{EX}), lowest observed concentration during the 24-hrs (Nadir).

TABLE 10
Nonlinear Dynamics of GH and HRVEP During Exercise and Resting Conditions.

| | | ACF | AMI | m | REC | DET | RATIO | Lₘₐₓ | SampEn |
|---|---|---|---|---|---|---|---|---|---|
| Exercise | GH | 4.6* ± 1.4 | 3.0† ± 1.0 | 9.1 ± 2.2 | 0.13 ± 0.11 | 0.91† ± 0.06 | 23.27 ± 29.23 | 42.00 ± 23.44 | 0.40 ± 0.17 |
| | SDNN_{EP} | 3.0 ± 2.0 | 1.0 ± 0.0 | 8.0 ± 1.5 | 0.26 ± 0.13 | 0.97 ± 0.03 | 5.51 ± 5.30 | 53.71 ± 22.66 | 0.58 ± 0.05 |
| | rMSSD_{EP} | 7.0 ± 4.9 | 1.1 ± 0.4 | 8.1 ± 1.5 | 0.23 ± 0.27 | 0.90 ± 0.19 | 23.40 ± 34.28 | 49.86 ± 46.85 | 0.54 ± 0.14 |
| | SampEn_{EP} | 1.0 | 1.0 | 7.6 ± 1.7 | 0.34 ± 0.17 | 0.98 ± 0.01 | 3.73 ± 2.14 | 42.00 ± 26.03 | 0.79 ± 0.06 |
| Rest | GH | 7.1* ± 1.1 | 5.9† ± 1.8 | 9.7 ± 1.3 | 0.15 ± 0.14 | 0.89† ± 0.12 | 12.40 ± 11.31 | 32.71 ± 20.25 | 0.46 ± 0.17 |
| | SDNN_{EP} | 4.3 ± 7.4 | 1.0 ± 0.0 | 8.1 ± 1.8 | 0.19 ± 0.13 | 0.96 ± 0.04 | 11.27 ± 12.12 | 34.86 ± 21.84 | 0.61 ± 0.11 |
| | rMSSD_{EP} | 10.1 ± 9.1 | 1.3 ± 0.5 | 6.4 ± 1.1 | 0.34 ± 0.30 | 0.86 ± 0.16 | 5.43 ± 6.10 | 58.43 ± 42.58 | 0.59 ± 0.17 |
| | SampEn_{EP} | 1.0 | 1.0 | 8.0 ± 0.8 | 0.36 ± 0.08 | 0.98 ± 0.005 | 2.89 ± 0.80 | 49.71 ± 9.96 | 0.77 ± 0.11 |

(n = 7) Data presented as mean ± sd
†p < 0.05;
*p < 0.1.
The mean ± sd autocorrelation function (ACF), average mutual information (AMI), optimal-embedding dimension (m), recurrence (REC), determinism (DET), ratio of DET/REC (RATIO), length of the longest diagonal line (Lₘₐₓ), and sample entropy (SampEn) values for the 24-hr GH profile and the HRV_{EPdata} data; standard deviation of the normal RR-intervals (SDNN); root mean square of successive differences of the normal RR-interval (rMSSD) and SampEn.

The parameter associated with the dynamics of each of the profiles (GH, SDNN_{EP}, rMSSD_{EP}, and SampEN_{EP}), as well as the quantification of the recurrence plots for these profiles are presented in Table 10. The multivariate model indicated a significant difference in the optimal parameters (time-lag and embedding dimension) for the nonlinear assessment of the GH profiles between conditions after controlling for changes in BF, FFM, and VO2max (V=0.81, F_{(1,3)}=12.57, p=0.04). Univariate tests indicated a near significant difference in the optimal time-lag, as calculated by the ACF (F_{(1,3)}=8.29, p=0.06) and AMI (F_{(1,3)}=8.36, p=0.06), for the exercise and resting conditions. The optimal embedding dimension was also different between conditions relative to changes in FFM (F_{(1,3)}=32.04, p=0.01).

The multivariate model did not indicate any significant differences between conditions for the optimal nonlinear dynamics parameters (V=0.08, F_{(1,3)}=0.27, p=0.64), or the dynamics (REC, DET, SampEn) of the SDNN_{EP} profile (029, F_{(1,3)}=1.22, p=0.35). While the dynamics of the multivariate model testing differences in the dynamics of rMSSD_{EP} were not significantly different between models (V=0.06, F_{(1,3)}=0.16, p=0.72), exploratory follow up tests indicated a significant difference in the REC of rMSSD_{EP} be-tween conditions (F_{(1,3)}=26.72, p=0.01) after controlling for changes in BF, FFM, and VO2max. Neither the dynamics (V=0.007, F_{(1,3)}=0.02, p=0.88), or optimal parameters (V=0.07, F_{(1,3)}=0.23, p=0.56) were different between conditions after controlling for the change in BF, FFM, and VO2max.

The values for joint-entropy and mutual information for GH-SDNN_{EP}, GH-rMSSD_{EP}, and GH-SampEn_{EP} are presented in Table 11. The multivariate model indicated non-significant differences in the joint-entropy measures between exercise and resting conditions (V=0.22, F_{(1,3)}=0.85, p=0.43) after controlling for changes in BF, FFM, and VO2max. However, the exploratory univariate tests did indicate a significant interaction between changes in FFM and condition for the joint-entropy of GH-SampEn_{EP} (F_{(1,3)}=10.87, p=0.04). Mutual information between GH-SDNN_{EP}, GH-rMSSD_{EP}, and GH-SampEn_{EP} were not significantly different between conditions (V=0.48, F_{(1,3)}=2.80, p=0.19).

TABLE 11
Joint-Entropy and Mutual Information During Exercise
and Resting Pro-files for GH-HRV_{EP} Data.

| | Joint entropy | | | Mutual information | | |
|---|---|---|---|---|---|---|
| | GH- | | | GH- | | |
| | SDNN_{EP} | rMSSD_{EP} | SampEn_{EP} | SDNN_{EP} | rMSSD_{EP} | SampEn_{EP} |
| Exercise | 2.65 ± 0.35 | 2.67 ± 0.26 | 2.78 ± 0.25 | 0.22 ± 0.06 | 0.26 ± 0.07 | 0.20 ± 0.05 |
| Rest | 2.66 ± 0.47 | 2.69 ± 0.29 | 2.79 ± 0.24 | 0.23 ± 0.06 | 0.27 ± 0.08 | 0.20 ± 0.03 |

(n = 7) Data presented as mean ± sd
Measures calculated as GH and standard deviation of the normal RR-interval (SDNN); root mean square of successive differences (rMSSD); sample entropy (SampEn).

Analysis from the cRQA plots is presented in Table 12. Multivariate models testing the differences between REC, DET, Lmax, entropy, and entropyNL were performed for each of the following crossed-profiles: GH-SDNN_{EP}, GH-rMSSD_{EP}, GH-SampEn_{EP}, GH-cortisol, cortisol-SDNN_{EP}, cortisol-rMSSD_{EP}, cortisol-SampEn_{EP}, SDNN_{EP}-SampEn_{EP}, and rMSSD_{EP}-SampEn_{EP}. None of these indices were different between conditions (p value range=0.66-0.98). However, the change in FFM was significantly associated with condi-tion after controlling for the change in BF and VO2max for GH-SDNN_{EP} (V=0.66, F_{(1,3)}=5.72, p=0.09), GH-rMSSD_{EP} (V=0.72, F_{(1,3)}=7.80, p=0.06), and cortisol-SDNN_{EP} (V=0.66, F_{(1,3)}=5.86, p=0.09). Similarly, the change in VO2max (V=0.80, F_{(1,3)}=12.0, p=0.04) significantly contributed to the model for GH-cortisol. Notable findings from the exploratory follow-up tests included a significant difference between conditions for entropy_{NL} of GH-SampEn_{EP} (F_{(1,3)}=7.1, p=0.076), the Lmax (F_{(1,3)}=7.84, p=0.067), entropy (F_{(1,3)}=17.86, p=0.02), and entropy_{NL} (F_{(1,3)}=6.56, p=0.08) of cortisol-rMSSD_{EP}, the entropy_{NL} (F_{(1,3)}=12.54, p=0.038) of cortisol-SampEn_{EP}, and entropy_{NL} (F_{(1,3)}=7.84, p=0.067) of rMSSD_{EP}-SampEn_{EP}.

TABLE 12
cRQA Analysis of the Biomarker and HRV_{EP} Profiles.

| | Exercise | | | | | Rest |
|---|---|---|---|---|---|---|
| | REC | DET | Lₘₐₓ | Entropy | Entropy_{NL} | REC |
| GH- | 55.70 ± 4.57 | 86.64 ± 6.28 | 20.86 ± 11.48 | 1.71 ± 0.19 | 0.65 ± 0.13 | 56.25 ± 8.85 |
| SDNN_{EP} | | | | | | |
| GH- | 53.32 ± 6.42 | 94.14 ± 3.55 | 40 ± 15.07 | 2.02 ± 0.26 | 0.57 ± 0.08 | 60.19 ± 8.04 |
| rMSSD_{EP} | | | | | | |
| GH- | 68.90 ± 8.79 | 98.44 ± 1.55 | 61 ± 9.06 | 2.66 ± 0.16 | 0.65* ± 0.04 | 71.75 ± 10.21 |
| SampEn_{EP} | | | | | | |
| GH- | 46.80 ± 3.81 | 83.17 ± 11.31 | 9.57 ± 2.15 | 1.38 ± 0.37 | 0.70 ± 0.14 | 47.86 ± 6.14 |
| Cortisol | | | | | | |
| Cortisol- | 55.61 ± 4.77 | 83.88 ± 6.64 | 10.14 ± 2.34 | 1.81 ± 0.34 | 0.83 ± 0.07 | 56.69 ± 6.43 |
| SDNN_{SP} | | | | | | |
| Cortisol- | 53.61 ± 6.19 | 91.76 ± 4.38 | 11.57* ± 0.79 | 1.88† ± 0.22 | 0.80* ± 0.06 | 60.26 ± 6.07 |
| rMSSD_{EP} | | | | | | |
| Cortisol- | 50.64 ± 9.08 | 82.53 ± 15.30 | 4.71 ± 0.96 | 0.45 ± 0.38 | 0.33† ± 0.26 | 45.50 ± 9.76 |
| SampEn_{EP} | | | | | | |
| SDNN_{EP}- | 55.75 ± 4.73 | 86.46 ± 6.06 | 17.57 ± 7.98 | 1.67 ± 0.20 | 0.65 ± 0.14 | 56.05 ± 8.98 |
| SampEn_{EP} | | | | | | |
| rMSSD_{EP}- | 52.88 ± 6.40 | 93.94 ± 3.91 | 35.57 ± 14.90 | 1.82 ± 0.19 | 0.53† ± 0.07 | 59.89 ± 8.39 |
| SampEN_{EP} | | | | | | |

(n = 7) Data presented as mean ± sd

| | Rest | | | |
|---|---|---|---|---|
| | DET | Lₘₐₓ | Entropy | Entropy_{NL} |
| GH- | 85.79 ± 8.05 | 21.14 ± 17.24 | 1.69 ± 0.36 | 0.66 ± 0.15 |
| SDNN_{EP} | | | | |
| GH- | 86.87 ± 12.49 | 41.86 ± 24.38 | 2.03 ± 0.46 | 0.60 ± 0.08 |
| rMSSD_{EP} | | | | |
| GH- | 96.63 ± 1.84 | 74.71 ± 29.18 | 3.13 ± 0.46 | 0.75* ± 0.03 |
| SampEn_{EP} | | | | |
| GH- | 87.77 ± 10.94 | 10.14 ± 1.46 | 1.28 ± 0.31 | 0.62 ± 0.16 |
| Cortisol | | | | |
| Cortisol- | 82.70 ± 6.99 | 9.14 ± 2.54 | 1.64 ± 0.30 | 0.81 ± 0.09 |
| SDNN_{SP} | | | | |
| Cortisol- | 87.35 ± 12.64 | 10.29* ± 1.60 | 1.62† ± 0.31 | 0.74* ± 0.10 |
| rMSSD_{EP} | | | | |
| Cortisol- | 85.15 ± 16.83 | 4.57 ± 1.62 | 0.56 ± 0.31 | 0.53† ± 0.31 |
| SampEn_{EP} | | | | |
| SDNN_{EP}- | 85.44 ± 8.37 | 19 ± 18.10 | 1.65 ± 0.36 | 0.71 ± 0.17 |
| SampEn_{EP} | | | | |
| rMSSD_{EP}- | 86.27 ± 13.31 | 37.14 ± 27.55 | 1.87 ± 0.51 | 0.62† ± 0.13 |
| SampEN_{EP} | | | | |

(n = 7) Data presented as mean ± sd
†p < 0.05;
*p < 0.1.
Growth hormone (GH); Variability and complexity indices calculated on the HRV_{EP} profiles: Quantification measures from cRQA: recurrence (REC); determinism (DET); maximal line length (Lₘₐₓ); Shannon-entropy of the lines longer than the minimum line length (Entropy); Entropy normalized to the number of lines (Entropy_{NL}).

One goal of this study was to delineate the difference in the dynamics of hypothalamic-pituitary regulation and cardiac control following an exercise perturbation compared to rest in healthy males over a 24-hr period. While not intending to be bound by theory, it is believed that a high-intensity exercise perturbation would result in changes in the regulatory dynamics of the hypothalamic-pituitary axis and cardiac-regulation—assessed through changes in GH and HRV. Specifically, it was hypothesized that the univariate and multivariate dynamics of these markers would be distinguishable from rest through nonlinear dynamics. The findings show that the effects of regulatory dynamics of the hypothalamic-pituitary axis and cardiac function following exercise are far from straightforward and that each of the different measures of HRV_{EP} appear to provide distinct information about the functionality of the system(s).

Cardiac control was assessed with continuous 24-hr RR-recordings and epoched-HRV data which consisted of 3-min windows analyzed every 10-min throughout the day. The 24-hr HRV data was consistent with previously established norms (214). While we don't have comparative data for the HRV_{EP} profiles, the values from the individual 3-min epochs were consistent with HRV findings from other short-time recordings (in the present study). Short-time RR-recordings are commonplace within the HRV literature and provide important information about cardiac control but can be affected by selection bias (i.e. acutely affected by physical, mental, or psychosocial stresses) whereas 24-hr RR-recordings can provide information about the overall state of the system (over this time frame). HRV_{EP} was performed to assess the patterned change in cardiac control throughout the 24-hr period.

However, these findings did indicate a significant difference in SDNN_{RR}, LF power, and SampEn_{RR} between exercise and rest whereas no differences were observed in SDNN_{EP}, rMSSD_{EP}, or SampEn_{EP}. From a physiological perspective, this suggests that although HRV_{EP} is a simple non-invasive method with low computation cost that provides a method of assessing acute and chronic changes in cardiac control throughout the day, it does not necessarily detect the same overall changes in cardiac control as a 24-hr recording. However, it does permit one to investigate how short-term regulation of cardiac control is being affected—which provides a method of tracking how changes in cardiac control are associated with changes in hypothalamic-pituitary regulation.

Interestingly, the dynamics of these HRV_{EP} profiles were not different between conditions, which suggests that while short-time HRV indices can be affected by acute stresses (represented by the dip in SDNN_{EP} and rMSSD_{EP} profiles—FIG. 5.3.A, 5.3.C), the dynamics of these HRV_{EP} time-series are robust against these acute perturbations (where indices of 24-hr HRV were not). Outside of the obvious effect of exercise on the SDNN_{EP} and rMSSD_{EP} profiles, the general upward trends during the nighttime hours was reproduced in both conditions. Similarly, SampEn_{EP} was very acutely affected by the exercise stimulus but returned to mean-stationarity almost immediately.

While the SDNN_{EP} and rMSSD_{EP} profiles are trend-stationary (detrending provides a stationary time-series), the stationary nature of the SampEn_{EP} data suggests that this time-series is more robust against raw changes in behavioral and environmental conditions throughout the day. The trends within SDNN_{EP} and rMSSD_{EP} appear to be deterministic properties associated within the physiologic response to changes in behavioral and environmental conditions throughout the day (i.e. sleep). Each of these measures is associated with different characteristics of cardiac control and the stochastic nature of each of these HRV_{EP} profiles provide additional context to the physiologic phenomena occurring throughout the day. Nonsignificant differences in REC, DET, and SampEn of these profiles (SDNN_{EP}, rMSSD_{EP}, and SampEn_{EP}) between rest and exercise provides additional evidence for specific physiologic regulatory mechanisms of cardiac control, most of which are well described in the literature. Thus, there appears to be a very strong inherent attractor that regulates cardiac function (in healthy individuals).

In some cases, each of the HRV_{EP} profiles likely provide some context to the changes in cardiac control throughout the day, but that depending on the intended purpose of this information, each of these measures provide different information. Specifically, SDNN_{EP} and rMSSD_{EP} follow the general trend of the raw RR-recording rather closely which suggests that these profiles may provide some context to the underlying circadian regulation of cardiac control whereas SampEn_{RR} may provide better context to the overall health of the system.

While differences in weight, BF, FFM, and VO₂ₘₐₓ were not statistically different between conditions, changes in each of these measures are known to drastically affect GH output and secretory dynamics. Thus, the difference in BF, FFM, and VO₂ₘₐₓ from exercise compared to rest was included within each of the analyses. GH output, was consistent with previously reported values. Exercise increased the total GH_{AUC} from 1080 ng/ml*24-hrs to 1602 ng/ml*24-hrs and GH_{N-AUC} from 608 ng/ml/min to 702 ng/ml/min from rest to exercise respectively. Similarly, exercise caused a distinct GH response with a mean GH_{EX}-Peak response of 7.98 ng/ml.

After controlling for the differences in these measures, GHAUC, GHN-AUC, and GHN-Peak were still significantly elevated following an acute high-intensity exercise bout com-pared to rest. While total GH output has been shown to be similar following resistance exercise compared to rest during nighttime hours, the GH secretory dynamics were altered via an attenuation of GH burst mass and pulse amplitude and increase in pulse frequency. Repeated exercise bouts and long-duration aerobic exercise have been shown to stimulate GH secretion compared to resting conditions. The complexity of GH secretion (assessed via approximate entropy, computationally similar to SampEn) has also been shown to be elevated following exercise compared to rest. GH output dynamics (SampEnGH) was not different between conditions.

Further, a higher DET_{GH} was observed during exercise compared to rest relative to changes in VO₂ₘₐₓ and after controlling for changes in BF and FFM. This suggests that a change in fitness, even over the course of an 8-12-week period, can alter the determinism of GH output. Changes in fitness and body composition are known to alter GH output and GH secretory patterns. Thus, even small changes in these variables over relatively short time spans appears to impact the dynamics of the physiological system and how it responds to stimuli. From a practical perspective, these are important considerations for individuals interested in altering body composition or individuals undergoing treatment options that may alter body composition.

Consideration of the findings from cRQA that assessed the dynamics of GH-HRV_{EP} may suggest that the dynamics associated with the variability of cardiac control throughout the day (assessed through SDNN_{EP} and rMSSD_{EP}) either are, or are-not, closely tied to hypothalamic-pituitary control (assessed through GH). However, interpretation of the whole of these findings (specifically considering GH-SampEn_{EP} and cortisol-SampEn_{EP}) suggests that SampEn_{EP} provides a unique, more sensitive, perspective into the dynamics between hypothalamic-pituitary regulation compared to SDNN_{EP} or rMSSD_{EP} with either of these biomarkers. Nevertheless, some of the diurnal patterns observed with SDNN_{EP} and rMSSD_{EP} may be more closely tied to cortisol (a key output from the hypothalamic-pituitary-adrenal axis). The time-scales at which each of these measures (GH and cortisol) operate is drastically different and exercise inversely affected the relations between these profiles with respect to changes in cardiac complexity throughout the 24-hr period.

In summary, the relationships among these markers suggests that there may be crossover between hypothalamic-pituitary functioning, assessed via GH dynamics and cardiac regulation—supporting theoretical frameworks and complimenting evidence pro-vided by others. HRV has been linked to a number of psychosocial constructs including emotion, cognition, and self-regulation as well as hypothalamic-pituitary-adrenal axis functioning. While much remains to be elucidated about how these measures are altered in response to additional chronic and acute stimuli, these findings provide a foundation of evidence that supports the methods described herein and suggests that there is a shared attractor between these systems. Furthermore, considering how the pattern of different HRV_{EP} indices change with GH output throughout the course of a 24-hr period at rest and following exercise, assessing, and modeling these measures together provides the foundation for a larger scalable model that can extrapolate physiologic responses through different levels of hierarchy and predict these responses across time.

### Example 4

### Modeling Dynamic Regulatory Patterns Between Biomarkers of Hypothalamic-Pituitary Axis and Cardiac Control

Differences in cardiac-dynamics during daytime hours following rest and exercise are associated with the ability to utilize learning algorithms to predict changes in hypothalamic-pituitary function, as assessed via GH. As demonstrated herein, patterns of GH output can be predictable using measures of cardiac control as the learnable parameter. Data for creating the models described herein was acquired using the procedures and methods described in Example 3.

Autoregressive-moving-average models were performed using “nlme: Linear and Nonlinear Mixed Effects Models”. All machine-learning procedures were run using Keras with a Tensorflow backend.

A mixed-model with an autoregressive (AR) correlation structure was used to compare the linear relationships between indices of cardiac-control and serum cortisol with GH profiles throughout a 24-hr period of rest and following exercise. All data were person-level centered after taking the natural log of the difference of time-series. The common, first-order autoregressive model is defined as:

Yₜ=β₀+β₁Yₜ₋₁+uₜ

and the autoregressive model of p^{th} order is defined as:

Yₜ=β₀+β₁Yₜ₋₁+β₂Yₜ₋₂ . . . βₚYₜ₋ₚ+uₜ

where p is the number of lags. An additional model, assessing the effects of lagging the predictor variables was performed for comparison. This model was defined as:

Yₜ=β₀+β₁Yₜ₋₁+ . . . +βₚYₜ₋ₚ+δ₁Xₜ₋₁+δ₁Xₜ₋₁+ . . . +δᵣXₜ₋ᵣ+uₜ

where p represents the number of lags of Y and r is the number of lags of X.

Machine learning algorithms were used to predict GH output based on patterned change in cardiac control. Long-short-term-memory (LTSM) networks are a special case of recurrent neural networks designed to avoid the issues associated with long-term dependencies that aren't dealt with as well in ordinary recurrent neural networks.

Two separate LSTM model frameworks were tested. First, a univariate model using a lagged GH sequence was used to predict nighttime GH secretory patterns. Second, a multivariate model with two input layers, two hidden layers, and a single outcome measure using two time-steps was used to predict nighttime GH secretion following rest and exercise. Specifically, a lagged time-series of SampEn_{EP} and GH were used to predict future GH output. Training was performed on the first 14-hrs of the 24-hr profiles over 80 epochs. The exercise and rest profiles for each individual were run separately across five-iterations to compare model fit and reproducibility. Mean fit indices, including the root mean square of the error (RMS) and mean absolute error (MAE), were compared across conditions. Univariate repeated-measures analysis of variance (ANOVA) was used to test differences in fit indices between exercise and resting conditions.

The ARMA (model-1) established that GH output throughout the course of the 24-hrs was not different throughout the 24-hr period for either condition. Model-2 included rMSSD_{EP}, SampEn_{EP}, and cortisol. While there was not a significant difference between conditions, model-2 was a significant improvement over model-1 (p<0.001). Model parameters are provided in Table 13.

TABLE 13
GLS Mixed-Models

| | Model-1 | Model-2 |
|---|---|---|
| Condition | 0.000 ± 0.044 | 0.0005 ± 0.043 |
| rMSSD_{EP} | | −0.006 ± 0.025 |
| SampEn_{EP} | | −0.231‡ ± 0.075 |
| Cortisol | | −0.218‡ ± 0.028 |
| rMSSD_{EP}:SampEn_{EP} | | 0.073* ± 0.042 |
| rMSSD_{EP}:Cortisol | | −0.222‡ ± 0.030 |
| SampEn_{EP}:Cortisol | | 0.002 ± 0.017 |
| rMSSD_{EP}:SampEn_{EP}:Cortisol | | 0.052‡ ± 0.010 |
| Constant | −0.000 ± 0.031 | −0.011 ± 0.032 |
| N | 2,016 | 2,016 |
| Log Likelihood | −2,853.6 | −2,785.9 |
| Akaike Inf. Crit. | 5,713.1 | 5,591.9 |
| Bayesian Inf. Crit. | 5,729.9 | 5,648.0 |

Data are presented as mean ± se
‡p < 0.01;
† p < 0.05;
*p < 0.1
Log likelihood (LL); Akaike information criteria (AIC); Bayesian information criterion (BIC). Models significantly different (p < 0.001)

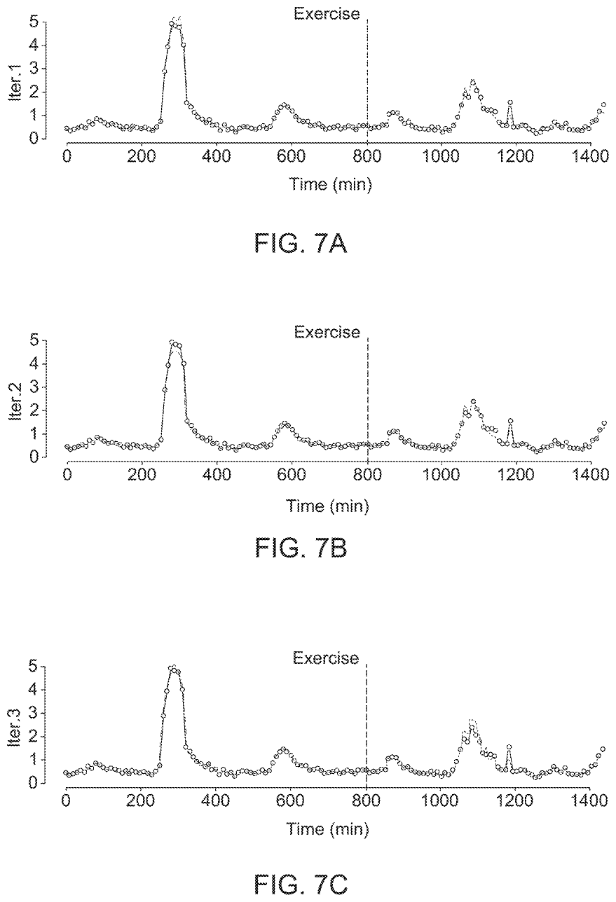

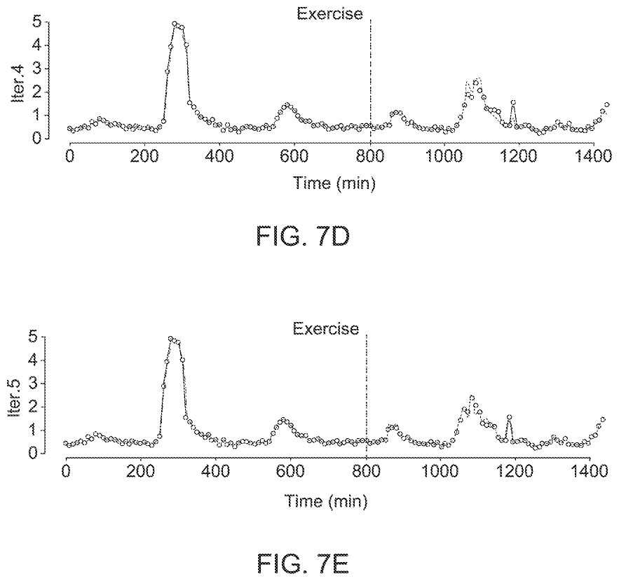

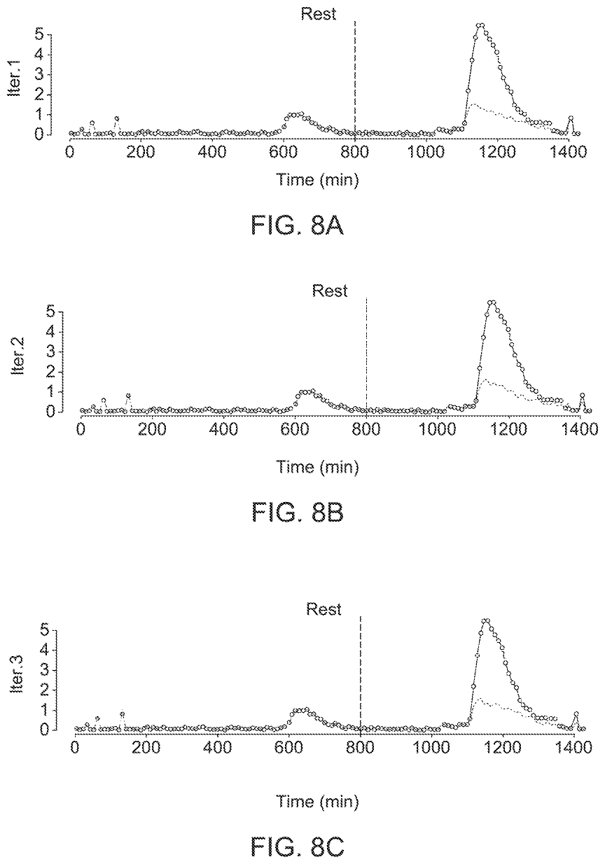

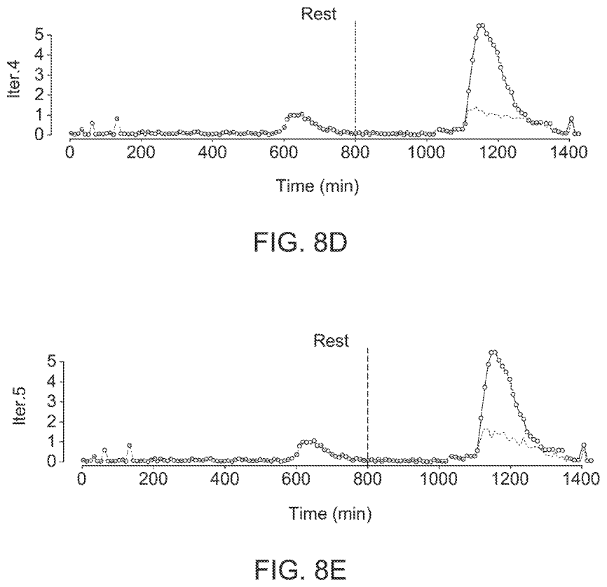

Mean fit indices (RMS and MAE) from the LSTM models are provided in Table 14. Comparison of these results indicated that RMS (F_{(1.6)}=9.46, p=0.02), but not MAE (F_{(1.6)}=0.004, p=0.95) was different between conditions. Backtesting was not performed on these data as the time-series were limited in data-length. Test-validation was per-formed by estimating the testing training datasets while simultaneously considering the loss-functions. Visual comparisons of predicted and actual data are provided FIGS. 7A-7E and FIGS. 8A-8E.

TABLE 14
Mean Fit from Five-Iterations of LSTM Networks
to the Exercise and Resting Profiles

| | | | |
|---|---|---|---|
| RMS | Exercise | 0.177 | ±0.192† |
| | Rest | 0.394 | ±0.216 |
| MAE | Exercise | 0.559 | ±0.728 |
| | Rest | 0.577 | ±0.192 |

Data presented as mean ± sd

†(p < 0.05) Comparisons made between conditions

Univariate repeated-measures ANOVA was used to test the within and between subject differences in the means of the fit indices (RMS) for the 5-iterations performed on each profile as outlined in the a priori statistical analysis section. However, additional information was included into these models in order to better understand what factors may have been associated with better- or worse-fitting models. Specifically, two sets of covariates were included (separately) into the model. The first set of covariates included the difference in body fat (BF_{diff}), the difference in fat-free mass (FFM_{-diff}), and the difference in VO₂ₘₐₓ (VO_{2max-diff}) between exercise and resting profiles. The second set of co-variates included the difference in the optimal embedding dimension for the 24-hr GH profile (Edim_{diff}), the difference in the determinism of the 24-hr GH profile (Maid and the difference in SampEn of the 24-hr GH profile (SampEn_{diff}) during the rest and exercise condition. Most notably, these analyses indicated a near significant interaction between DET_{diff} and condition (F_{(1,3)}=7.57, p=0.07) for RMS, indicating that a more deterministic 24-hr GH profile during the exercise condition was associated with a lower RMS value compared to rest after controlling for changes in BF and VO₂ₘₐₓ. In addition, an interaction among BF_{diff} and condition was observed (F_{(1,3)}=10.81, p=0.05), indicating that higher degrees of body fat during the exercise condition were associated with differences in RMS values between exercise and resting conditions.

FIGS. 7A-7E show good performing LSTM predictions models across 5-iterations of a single exercise profile over a 24-hr period. Actual (black circles) versus predicted daytime (left of dotted line) and nighttime (right of dotted line) GH throughout the 24-hr period. LSTM models were trained on data to the left of the dotted line and predicted onto the remaining hours left of the dotted line.

FIGS. 8A-8E show poor-performing LSTM prediction models across 5-iterations of a single resting profile over a 24-hr period. Actual (black circles) versus predicted daytime (left of dotted line) and nighttime (right of dotted line) GH throughout the 24-hr period. LSTM models were trained on data to the left of the dotted line and predicted onto the remaining hours left of the dotted line.

Initial mixed-models with an autocorrelation structure indicated that the addition of the predictor variables (rMSSD_{EP}, SampEn_{EP}, and cortisol) had a significant impact on model performance. This provided supporting evidence that these may be able to predict GH output based on changes in cardiac control (HRV_{EP}) and cortisol throughout the day. The model itself indicated a significant interaction among rMSSD_{EP}, SampEn_{EP}, and cortisol, which supports the idea that each of these indices may contribute different information to a larger machine learning model. More specifically, the individual contribution of SampEn_{EP} was notably higher than rMSSD_{EP} which supports previous hypotheses that these indices provide different information about the state of the system and that SampEn_{EP} may provide better estimative abilities compared to other HRV_{EP} indices. These findings also support the notion that cortisol may provide important information from a GH prediction standpoint and suggest that the significant contributions of cortisol within these models are related to the circadian regulation of cortisol.

Nevertheless, these finding supported the hypothesis that the patterns within the changes of cardiac complexity throughout the day could serve as a non-invasive marker to predict changes in GH output. Considering the findings from the mixed model with the autocorrelation structure, we then tested the ability of SampEn_{EP} to predict GH output through an LSTM network. These models were trained on the first 14-hrs of each individual time-series and tested on the nighttime hours. Preliminary models (data not shown) were fit to the univariate GH time-series, using single and double-lag. These models produced surprisingly modest results, however, these models were not included as our primary objective was to investigate the ability of changes in cardiac control to predict GH output.

The ability of these LSTM networks to predict GH output was significantly better with the exercise profiles compared to the resting profiles—as indicated by the lower RMS for exercise (Table 14). RMS gives a higher weight to larger errors than MAE and thus, because larger errors were particularly undesirable given the nature of the analytes, more emphasis was placed on changes in RMS compared to MAE. The effect of these larger errors on overall model performance can be easily observed in FIGS. 8A-8E where the predicted GH output was substantially lower than the observed values.

Though the exercise-models did sometimes prematurely anticipate the GH response (i.e. the models predicted the increase in GH incorrectly) or predict the peak or undershoot/overshoot the height of the GH peak, it rarely missed both. There were instances where the models produced highly variable estimates, even though the general patterns were the same which may have to do with stochastic gradient descent and disappearing gradients specific to that model. The weights of the parameters are updated by taking the derivative of the loss with respect to the parameter which is initialized randomly (in order to find the minimums). If these parameters are being chosen incorrectly, the weights/estimates of the subsequent layers (which are carried from layer to layer) become increasingly less accurate and the accuracy of the model decreases. Previous research has shown that the dynamics of univariate and multivariate profiles appear to be highly individualized and the inconsistent performance associated with some of these models further highlights that these dynamic relationships may not only be different between conditions but differentially affected by exercise across individuals. If the latter point is true, modeling these responses will require additional information. In some cases, addition of demographic information into the model may be appropriate, as well as completely different variables being fed into the model, more training data, or other different model structures.

Though the resting profiles were, for the most part, reproducible between each of the iterations, they were less accurate at predicting the actual GH output compared to the exercise conditions. Interestingly, these instances also seemed to coincide with the profiles where SDNN_{EP}, and rMSSD_{EP} were less variable throughout the course of the 24-hr period. This is interesting given that the determinism of the HRV_{EP} profiles was not different between conditions whereas GH was more deterministic during the exercise condition compared to the resting condition.

1. A method of predicting biomarker concentrations from changes in short-time cardiac dynamics comprising:
measuring heart rate variability of a subject at rest;
measuring heart rate variability of the subject during physical exertion;
identifying changes in the heart rate variability at rest verses during physical exertion, and
determining an estimated level of a biomarker present in the subject based on the identified changes.

2. The method of claim 1, wherein measuring heart rate variability comprises recording an electrocardiogram of the subject.

3. The method of claim 1, wherein measuring heart rate variability comprises measuring R-R intervals of the subject.

4. The method of claim 3, further comprising predicting future biomarker concentrations present in the subject based on fluctuations and/or patterns in the R-R interval for a given period of time.

5. The method of claim 1, wherein the biomarker is a regulatory biomarker.

6. The method of claim 5, wherein the regulatory biomarker comprises one or more of growth hormone, glucagon, insulin, nesfatin-1, galanin, cortisol, glucose, insulin-like growth factor 1, or any combination thereof.

7. The method of claim 5, wherein the biomarker is hypothalamic-pituitary function-related biomarker.

8. The method of claim 7, wherein the hypothalamic-pituitary function-related biomarker comprises a pituitary hormone comprising: a growth hormone, somatostatin, growth-hormone releasing hormone, follicle-stimulating hormone, adrenocorticotropic hormone, thyroid-stimulating hormone, luteinizing hormone, vasopressin, oxytocin, and/or gonadotropin-releasing hormone.

9. The method of claim 5, wherein the biomarker is regulated by changes in hypothalamic-pituitary hormones.

10. The method of claim 9, wherein the hypothalamic-pituitary hormones comprise a growth hormone, somatostatin, growth-hormone releasing hormone, follicle-stimulating hormone, adrenocorticotropic hormone, thyroid-stimulating hormone, luteinizing hormone, vasopressin, oxytocin, and/or gonadotropin-releasing hormone.

11. The method of claim 1, wherein the biomarker comprises one or more of glucagon, glucose, insulin, and cortisol.

12. The method of claim 1, wherein the estimated level of the biomarker is a blood or salivary concentration of the biomarker in the subject.

13. The method of claim 1, wherein the estimated level of the biomarker is determined non-invasively.

14. The method of claim 1, wherein examining changes in the heart rate variability comprises examining cardiac regulatory dynamics comprising time-domain, frequency-domain, and/or complexity (nonlinear dynamics) of heart rate variability.

15. The method of claim 1, further comprising estimating biomarker dynamics.

16. The method of claim 15, wherein biomarker dynamics comprises output and secretory dynamics of the biomarker.

17. The method of claim 15, wherein the biomarker dynamics comprises output/concentration, pulse amplitude, and/or pulse burst frequency based on patterns in R-R intervals.
